(19) United States
(12) Patent Application Publication
(10) Pub. No.: US 2007/0238140 A1
(43) Pub. Date: Oct. 11, 2007

(54) Method For Multiplex Bead-Based Assays Using Chemiluminescence and Fluorescence

(76) Inventors: Stephen L. Pentoney JR., Chino Hills, CA (US); David L. Yang, Orange, CA (US); Clarence Y. Lew, Irvine, CA (US)

Correspondence Address: BECKMAN COULTER INC.;C/O SHELDON MAK ROSE & ANDERSON, 100 East Corson Street, Third Floor, PASADENA, CA, 91103-3842, US

(21) Appl. No.: 11/279,070

(22) Filed: Apr. 7, 2006

(51) Int. Cl. G01N 33/551

(52) U.S. Cl. 435/7.92; 436/524

(57) ABSTRACT

A method for detecting a target analyte in a sample, the method having the steps of: binding the target analyte to a fluorescently coded particle capable of specifically binding to the target analyte; labeling the target analyte with a first chemiluminescence component; adding a second chemiluminescence component to the labeled target analyte to produce chemiluminescence; stabilizing the particle; exciting fluorescence from the fluorescently coded particle; detecting fluorescence from the fluorescently coded particle; and detecting the chemiluminescence.

### BACKGROUND

The present invention relates to a detection system and method for measuring analytes bound to particles encoded with fluorescent labels.

Particles such as beads, microparticles, microholders, and microspheres are useful analytical tools for detecting and measuring various analytes, especially when combined with flow cytometry systems and methods. A multiplexed assay allows more than one analyte to be analyzed simultaneously. Multiplexing can be done using particles.

Analytes of interest are often bound to a particle and identified by a corresponding characteristic of the particle, such as fluorescence at one or more wavelengths, along with a fluorescent reporter label bound to the analyte. Examples of prior art assays using particles include: U.S. Pat. No. 3,925,018, Great Britain Patent No. GB1561042, Canadian Patent No. 1248873, and U.S. Pat. No. 6,859,570.

However, these systems suffer from low levels of reporter label fluorescence as well as background interference from Raman and Rayleigh scatter. Moreover, the use of multiple fluorescent labels can be disadvantageous when separate wavelength regions are not reserved for the emission spectra of the labeled analyte of interest and for the fluorescent particle labels. Overlapping emission spectra between analyte labels and particle labels can hinder detection and quantification of the analyte in these systems.

A need exists for an analytical detection system employing particle technology that can distinguish between multiple subpopulations of particles in a cost and time efficient manner while simultaneously accurately identifying and quantifying low levels of multiple analytes.

### SUMMARY

Accordingly, the present invention is directed to a method for detecting a target analyte in a sample comprising the steps of: binding the target analyte to a fluorescently coded particle capable of specifically binding to the target analyte; labeling the target analyte with a first chemiluminescence component; adding a second chemiluminescence component to the labeled target analyte to produce chemiluminescence; stabilizing the particles; exciting fluorescence from the fluorescently coded particle; detecting fluorescence from the fluorescently coded particle; and detecting the chemiluminescence.

In an embodiment, either the first chemiluminescence component or the second chemiluminescence component is a catalyst. Examples of a catalyst are enzymes, such as horseradish peroxidase, alkaline phosphatase and galactosidase, and metal ions, such as Fe⁺², Fe⁺³, Cu⁺ and Cu⁺². Optionally, chemiluminescence is detected prior to exciting and detecting fluorescence. Optionally, the particles are immobilized prior to adding the second chemiluminescence component.

The present invention is also directed to a kit for detecting a target analyte in a sample. In an embodiment, the kit comprises: a plurality of particles fluorescently coded for identification, each particle having at least one capture probe bound thereto, each capture probe being specifically bindable to the target analyte. The kit also comprises a labeling reagent bindable to the target analyte, the labeling reagent having a first chemiluminescence component. The kit further contains a second chemiluminescence component reactable with the first chemiluminescence component, to generate chemiluminescence.

The kit can be used by adding a sample containing the target analyte to the plurality of fluorescently coded particles at conditions such that the target analyte in the sample binds to the capture probes bound to the particles. This forms capture probe/target complexes. The labeling reagent is then added at conditions such that the labeling reagent binds to the target in the capture probe/target complex. The second chemiluminescence component is then added to generate chemiluminescence. The particles are stabilized, such as by allowing them to settle or by immobilizing them, and chemiluminescence and fluorescence are measured.

In another embodiment, the present invention is directed to a kit for detecting a plurality of target analytes in a sample, the kit having a plurality of sets of fluorescently coded particles, each set comprising particles having at least one capture probe bound thereto, and each set having a capture probe capable of binding with a different target analyte. The kit also has a plurality of different labeling reagents bindable to the target analytes, the labeling reagents being labeled with a first chemiluminescence component. The kit also has a second chemiluminescence component reactable with a first chemiluminescence component bound to the target analyte to generate chemiluminescence.

In an additional embodiment, the present invention is directed to a method for detecting a plurality of different target analytes in a sample comprising the steps of: binding each of the target analytes to a respective particle specific to the target analyte, each particle being fluorescently coded for identification; labeling each of the target analytes bound to the particles with a first chemiluminescence component; adding a second chemiluminescence component to the labeled target analytes to produce chemiluminescence; stabilizing the particles; exciting fluorescence from the fluorescently coded particles; detecting fluorescence from the fluorescently coded particles; detecting chemiluminescence; and associating detected fluorescence with detected chemiluminescence to determine the identity and presence of one or more of the target analytes.

In an additional embodiment, the present invention is directed to a method for detecting a target analyte in a sample comprising the steps of: mixing the target analyte with a plurality of fluorescently coded particles bindable to the target analyte; labeling the fluorescently coded particles having no target analyte with a first chemiluminescence component; adding a second chemiluminescence component to the labeled fluorescently coded particles to produce chemiluminescence; stabilizing the particles; exciting fluorescence from the fluorescently coded particle; detecting fluorescence from the fluorescently coded particle; and detecting the chemiluminescence.

### BRIEF DESCRIPTION OF THE DRAWINGS

These features, aspects and advantages of the present invention will become better understood with regard to the following description, appended claims and accompanying drawings where:

FIG. 1 is a flowchart of a method of detecting an analyte according to an embodiment of the present invention;

FIG. 2 is a schematic diagram of a system for detecting an analyte according to an embodiment of the present invention;

FIG. 3 is a schematic diagram of a system for detecting chemiluminescence using a plate reader according to an embodiment of the present invention;

FIG. 4 is a schematic diagram of a system for detecting fluorescence using a plate reader according to an embodiment of the present invention;

FIG. 5 is a photograph showing chemiluminescence detected from a mixture of HRP-modified intensely-stained beads and unmodified dimly-stained beads following addition of Lumigen PA-atto solution and after the beads were settled;

FIG. 6 is a photograph showing fluorescence detected from a mixture of HRP-modified intensely-stained beads and unmodified dimly-stained beads following addition of Lumigen PA-atto solution and after the beads were settled;

FIG. 7 is a photograph showing an overlay of the images of FIGS. 5 and 6 showing detected chemiluminescence and fluorescence;

FIG. 8 is a photograph showing chemiluminescence detected from a mixture of intensely-stained beads having an HRP labeled target bound to an antibody capture probe and unmodified dimly-stained beads, following addition of Lumigen PA-atto solution and after the beads were settled;

FIG. 9 is a photograph showing fluorescence detected from a mixture of intensely-stained beads having an HRP labeled target bound to an antibody capture probe and unmodified dimly-stained beads, following addition of Lumigen PA-atto solution and after the beads were settled; and

FIG. 10 is a photograph showing an overlay of the images of FIGS. 8 and 9 showing detected chemiluminescence and fluorescence.

### DETAILED DESCRIPTION

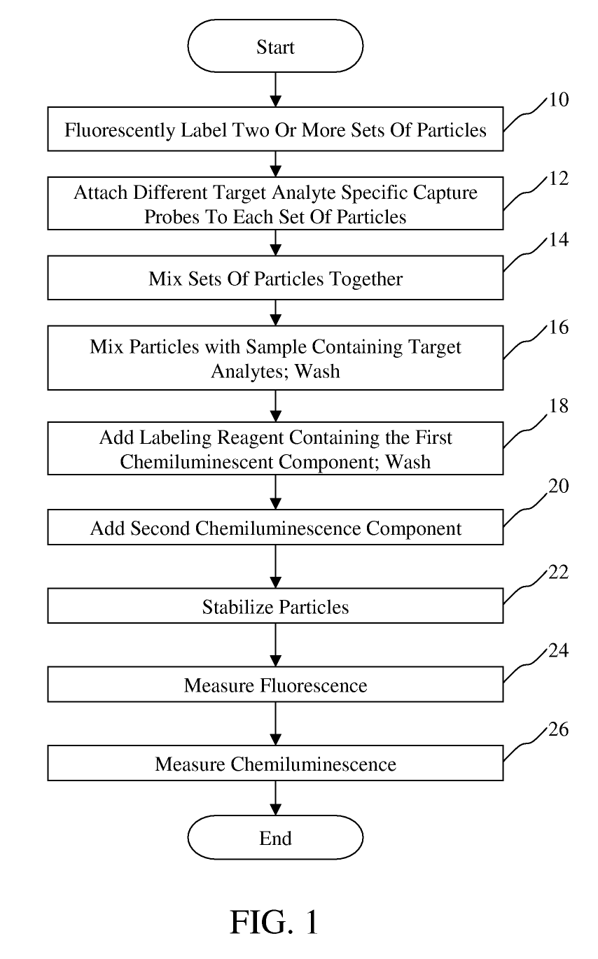

An overview of a method for detecting a target analyte according to an embodiment of the present invention is shown in FIG. 1. As shown in FIG. 1, two or more sets of particles are given different fluorescent labels, box 10. A different capture probe, such as an antibody, is attached to each set of particles, box 12. The different sets of particles are mixed together, box 14. The mixed particles are exposed to a sample that may contain at least one target analyte that binds to the capture probe on the appropriate particle, box 16.

The particles are exposed to a solution containing a labeling reagent, such as secondary antibodies, bindable to the possible target analytes being assayed for, box 18. The labeling reagents contain a first chemiluminescence component. A second chemiluminescence component is added to the particles, box 20. The particles are stabilized, for example by allowing the particles to settle to the bottom of a well in a microtiter plate, box 22. Using a light source, the fluorescent labels of the particles are detected to determine which target analytes are being tested for, box 24. Chemiluminescence is then measured to quantify target concentrations, box 26.

Preferably, the fluorescence excitation light is absent during the detection of chemiluminescence, thereby eliminating possible interfering Rayleigh scatter, Raman scatter, or fluorescence. This allows for long integration times and improved analyte sensitivity. Preferably the particles are kept stationary during measurement of chemiluminescence and fluorescence. However, some motion is acceptable as long as it can be ascertained from which particle each chemiluminescent signal arose.

As used herein, “capture probes” generally refer to materials that recognize and bind to target analytes in a sample. Capture probes also recognize and bind to controls.

As used herein, “sample” generally refers to a substance that is being assayed for the presence of one or more target analytes. For example, a sample may be taken using methods known in the art from a cell source or body fluid. Non-limiting examples of cell sources available in clinical practice include blood cells, buccal cells, cervicovaginal cells, epithelial cells from urine, fetal cells, or cells present in tissue obtained by biopsy. Body fluids can include blood, blood plasma, urine, cerebrospinal fluid, semen, tissue exudates, saliva, urine and fecal materials.

The capture probes, labeling reagents, and target analytes may be at least in part, for example, biomolecules such as nucleic acids, polynucleotides, proteins (i.e., an amino acid sequence containing more than 50 amino acids), and peptides (i.e., an amino acid sequence comprising fewer than 50 amino acids); cells and cellular components such as membrane receptors; biomolecule recognition sites, suborganelles, and other structural features. Of particular importance are proteins, including antibodies, antigens, enzymes, receptors, and small compounds such as peptides.

Polynucleotides may be DNA, RNA, or a DNA analog, such as PNA (peptide nucleic acid). The DNA may be a single- or double-stranded DNA, or a DNA amplified by PCR technique. The RNA may be an mRNA. The length of the polynucleotides may be from about 20 bp to about 10 kb. When the target is a polynucleotide, the capture probe and labeling reagent may comprise a polynucleotide that is complementary to the target polynucleotide or a portion thereof.

Typically, the capture probes are a monoclonal or polyclonal antibody to the target analyte or a portion thereof, such as a Fab fragment, which may specifically bind to the target analyte. However, as one skilled in the art can readily appreciate, the formation of a conjugate comparable to the binding of an antibody to an antigen may be achieved through the use of another protein- or peptide-based binding system, specific or nonspecific, such as a receptor protein or fragment thereof and a ligand therefore, which would not generally be considered to involve an immunochemical conjugation. Further, analogs and variants or mimics of various immunoreactants, such as those generated using recombinant DNA techniques, which bind to the target analyte, are contemplated to be within the scope of the present invention.

The particles or beads themselves may be commercially available polystyrene latex particles which are functionalized to permit the attachment of binding agents or capture probes for the target analytes of interest and indicators or signaling agents for indicating the occurrence of binding or reaction with the analyte of interest. Also possible are acrylate or methacrylate derived particles produced by suspension or dispersion polymerization techniques. Other possible bead or particle sources include glass beads, hydrogel polymer particles, polymerized micelle particles, particles produced by grinding cast films, particles produced by photopolymerization of aqueous emulsions, and particles produced by solvent casting as described in U.S. Pat. Nos. 4,302,166 and 4,162,282, the entire contents of which are hereby incorporated herein by reference.

Particle sizes typically range from about 0.1 μm to about 50 μm, and preferably from about 1 μm to about 20 μm. Particle density is typically in the range of 0.5 to 2.0 grams per milliliter. This combination of size and density permits aqueous suspensions of the particles to be handled as simple liquids which can be conveyed by a fluid transfer apparatus such as a pipette, pump, and/or a valve. Thus, the particles are freely transportable in aqueous suspension by conventional fluid transfer techniques.

It may be advantageous for the particles to include means (such as similar charges or surfactants) that effectively prevent interaction with other particles. This includes both prevention of the formation of particle agglomerates, which could clog the apparatus, and interference between particles of different types.

Each type of particle incorporates coding indicia that enables unambiguous identification of the particle type, and consequently enables the analysis system to assign measurement signals from the particle to the specific analyte with which the particle interacts. Particle labeling or coding can be accomplished by varying detectable particle properties such as intensity of fluorescence from fluorescent dyes associated with the particles, ratios of intensities of fluorescence from multiple fluorescent dyes associated with the particles, fluorescence wavelengths of one or more fluorescent dyes associated with the particles, and combinations of any of the above characteristics.

The materials and methods that are used to prepare the particles are well known in the art. The particles can be characterized by fluorescent dye attached onto the surface of the particle by standard surface chemistries via biomolecule bridges such as biotin-streptavidin, oligonucleotide, proteins or peptides after particle casting. Particles can also be labeled using a swelling/shrinking process in the presence of the desired fluorophore. This method is described by L. B. Bangs (Uniform Latex Particles; Seragen Diagnostics Inc. 1984, p. 40) the entire contents of which is incorporated herein by reference.

Known fluorescent dyes can be used to label individual sets of particles for identification. Examples of suitable fluorescent dyes which are hydrophobic and stable and can be used for this purpose include ([2-[2-[3-[(1,3-dihydro-3,3-dimethyl-1-propyl-2H-indol-2-ylidine)ethylidine]-2-(phenylthio)-1-cyclohexen-1-yl]ethenyl]-3,3-dimethyl-1-propylindolium perchlorate])(also known as IR792), ([2-[2-[3-[(1,3-dihydro-3,3-dimethyl-1-propyl-2H-indol-2-ylidine)ethylidine]-2-phenoxy-1-cyclohexen-1-yl]ethenyl]-3,3-dimethyl-1-propylindolium perchlorate]) (also known as IR768), ([2-[2-[3-[(1,3-dihydro-3,3-dimethyl-1-propyl-2H-benzoindol-2-ylidine)ethylidine]-2-(phenylthio)-1-cyclohexen-1-yl]ethenyl]-3,3-dimethyl-1-propylbenzoindolium iodide]), ([2-[2-[2-chloro-3-[(1,3-dihydro-3,3-dimethyl-1-propyl-2H-indol-2-ylidine)ethylidine]-1-cyclohexen-1-yl]ethenyl]-3,3-dimethyl-1-propylindolium perchlorate]) (also known as IR780), ([2-[2-[2-chloro-3-[(1,3-dihydro-3,3-dimethyl-1-decanyl-2H-benzoindol-2-ylidine)ethylidine]-1-cyclohexen-1-yl]ethenyl]-3,3-dimethyl-1-decanylbenzoindolium iodide]), ([2-[2-[-3-[(1,3-dihydro-3,3-dimethyl-1-decanyl-2H-benzoindol-2-ylidine)ethylidine]-1-cyclohexen-1-yl]ethenyl]-3,3-dimethyl-1-decanylbenzoindolium iodide]) and their derivatives. Individual dyes or combinations of dyes in varying amounts or ratios can be used to provide a unique identifier for each type of particle in the particle mixture.

In addition to the use of fluorescence, different categories of particles can be further distinguished by additional coding characteristics, for example by size, density, radioactivity, color, brightness, electrical charge, or magnetic properties.

The inventive method of attaching target analytes to the particles and of detecting target analytes will now be considered in greater detail. Contacting capture probes with target analytes (for example, hybridization) is conducted under conditions that allow the formation of stable complexes between the probes and the targets. For example, when target antigens are contacted with target specific primary antibodies, bound directly to the particles, the antigens bind to the primary antibodies, forming a capture probe/target complex.

As a second example, in polynucleotide assays, when target polynucleotides are contacted with capture probe polynucleotides bound to the particle, complementary regions on the target and the probe polynucleotides anneal to each other, forming a capture probe/target complex.

The selection of such conditions is within the level of skill in the art and includes those in which a low, substantially zero, percentage of mismatched hybrids form. The precise conditions depend, however, on the desired selectivity and sensitivity of the assay. Such conditions include, but are not limited to, the hybridization temperature, the ionic strength and viscosity of the buffer and the respective concentrations of the target analytes and capture probes. Hybridization conditions may be initially chosen to correspond to those known to be suitable in standard procedures for hybridization to filters and then optimized for use with the substrates of the present invention. The conditions suitable for the hybridization of one type of target material would appropriately be adjusted for use with other target materials.

In an embodiment, antigens are hybridized to primary antibodies bound to a particle surface. This may be done, for example, using a shaker at temperatures ranging from about 20° C. to about 50° C., for a period of from about 1 hour to about 24 hours, in a suitable hybridization buffer. A typical hybridization buffer contains phosphate buffered saline (PBS), typically in the range of from about 5 to about 30 mM, a blocker such as bovine serum albumin, in the range from about 0.5 to about 5.0%, and a surfactant such as Tween20, in the range from about 0.01 to about 1.0%.

Once capture probe/target complexes are formed, the substrates are washed under conditions suitable to remove non-specifically bound and free target biomolecules. Washing may be carried out at temperatures ranging from about 20° C. to about 50° C. with a buffer containing PBS and a surfactant. Preferably, the washing is carried out at room temperature with a buffer having 10 mM PBS, pH 7.4 and 0.05% Tween20.

In another embodiment, target polynucleotides are hybridized to probe polynucleotides at temperatures in the range of from about 20° C. to about 70° C., for a period of from about 1 hour to about 24 hours, in a suitable hybridization buffer. Suitable hybridization buffers for use in the practice of the present invention generally contain a high concentration of salt. A typical hybridization buffer contains in the range of from about 2× to about 10× SSC and from about 0.01% to about 0.5% SDS at pH 7-8.

Again, once capture probe/target complexes are formed, the substrates are washed under conditions suitable to remove non-specifically bound and free target biomolecules. Preferably, the washing is carried out at a temperature in the range of from about 20° C. to about 70° C. with a buffer containing from about 0.1× to about 2×SSC and from about 0.01% to about 0.1% SDS. The most preferred wash conditions for polynucleotides presently include a temperature that is the same as the hybridization temperature, and a buffer containing 2×SSC and 0.01% SDS. As previously noted, it would be a routine matter for those working in the field to optimize the contacting conditions for any given combination of target analyte and capture probes.

The bound-target is either directly or indirectly labeled with a first chemiluminescence component, via the labeling reagent, to facilitate detection. Preferably, the target is either directly or indirectly labeled with a chemiluminescent catalyst such as horseradish peroxidase, alkaline phosphatase, or galactosidase, or metal ions such as Fe⁺², Fe⁺³, Cu⁺ and Cu⁺².

In a preferred embodiment, a labeling reagent, such as a secondary antibody specific to a target analyte containing a catalyst, is hybridized to the target analyte, thereby labeling the target analyte for detection. The catalyst may be directly coupled to the secondary antibody or the attachment may be made indirectly, such as via a biotinylated secondary antibody and a streptavidin-catalyst.

In an alternative embodiment of the present invention, the labeling reagent may interact with unoccupied capture probes on the particle, as in a conventional competitive binding assay. In this embodiment, the labeling reagent is an analogue of the target analyte and is coupled to the first chemiluminescence component.

Additionally, in polynucleotide assays, a labeling reagent, such as a second polynucleotide, labeled with a catalyst, is contacted with the capture probe/target complexes. Complementary regions on the target and the second polynucleotide anneal to each other, thereby labeling the target analyte for detection.

The first chemiluminescence component acts with a second chemiluminescence component to produce chemiluminescence. The second chemiluminescence component may be a chemiluminescent substrate such as, for example, CDP-Star, CSPD, and Galactron substrates from Applied Biosystems in Foster City, Calif.; ECL reagents from Amersham Biosciences in Piscataway, N.J.; Super Signal substrate from Pierce Biotechnology, Inc. in Rockford, Ill.; Lumi-Phos, Lumigen APS, Lumigen PS, Lumigen PS-Atto, and Lumi-Gal 530 from Lumigen, Inc. in Southfield, Mich.; and LumiGLO from KPL, Inc. in Gaithersburg, Md.

Alternatively, the first chemiluminescence component directly or indirectly bound to the target analyte may be a chemiluminescent substrate. A catalyst is then added to the particles to generate chemiluminescence.

In a preferred embodiment of the present invention, the particles are allowed to settle on the bottom of a microplate after addition of the second chemiluminescence component. Additionally, in another embodiment, the particles can be immobilized prior to, during, or after addition of the labeling reagent containing the second chemiluminescence component.

It is known to immobilize microparticles by, for example, embedding them in glass fiber filters (e.g. U.S. Pat. Nos. 5,356,785 and 5,879,881, hereby incorporated by reference), sedimenting them on the bottoms of microtiter wells, packing them into microchannels, and attaching them to planar glass substrates (e.g., U.S. Pat. No. 6,133,436, hereby incorporated by reference). Microparticles have also been film-immobilized (e.g. U.S. Pat. Publ. No. 20030129296, the entire contents of which are hereby incorporated by reference).

In an embodiment of the present invention, the particles are immobilized in a chamber having pockets that capture the particles in a spaced relationship for further analysis. A device for immobilizing particles in pockets is disclosed in U.S. Pat. Publ. No. 20040096977, the entire contents of which are hereby incorporated by reference.

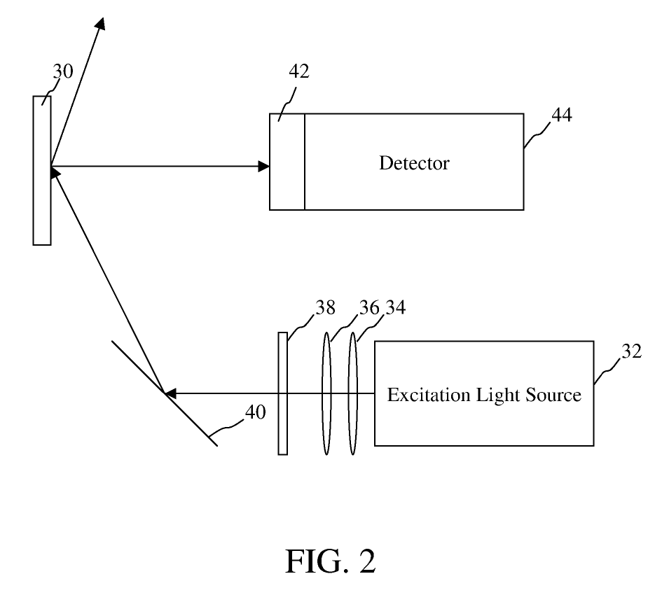

A detection system according to an embodiment of the present invention is shown in FIG. 2. Once the particles are settled or have been immobilized in a chamber 30, the particles are interrogated for both the identity of the particles, via fluorescence from the particle, and the presence and quantity of any target analyte, via chemiluminescence.

Detection of fluorescence is made using an excitation light source 32 coupled with appropriate optical manipulation equipment, such as lenses 34, 36, a filter 38, and a mirror 40. The excitation light source may be, for example, a laser. The excitation light excites the particles to fluoresce. Fluorescence emitted by the particles passes through optical manipulation equipment, such as a bandpass filter 42 and into a detector 44. The detector may be, for example, a CCD camera, diode array, or photographic film.

Detection of chemiluminescence is made by combining the bound target analyte labeled with the first chemiluminescence component with a solution containing the second chemiluminescence component. The excitation light source is turned off. As the solution containing the second chemiluminescence component is mixed with the immobilized particles, chemiluminescence is generated. The generated light passes through the appropriate optical manipulating equipment, such as the filter 42 and into the detector 44. Again the detector can be a CCD camera, diode array, or photographic film. One skilled in the art will recognize that different filters 42 or detectors 44 may be used during detection of fluorescence and chemiluminescence.

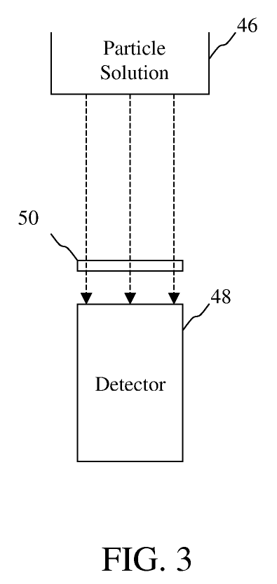

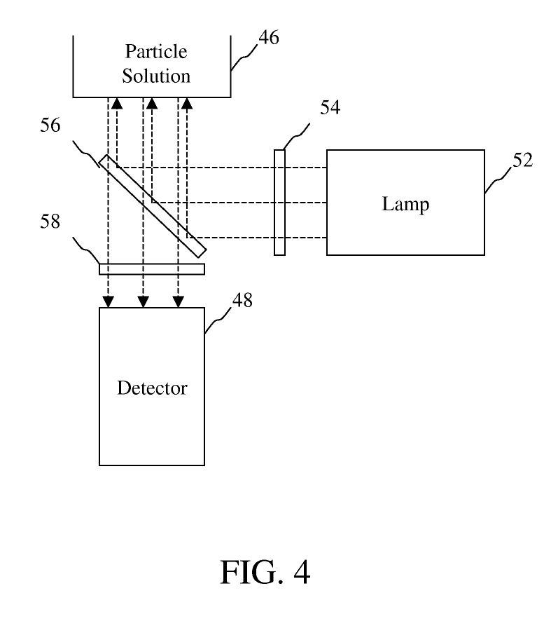

In a preferred embodiment, as shown in FIGS. 3 and 4, a plate reader with a lamp source is used for detecting fluorescence and chemiluminescence. For example, Beckman Coulter's A² (“A-squared”) plate reader can be used. A solution of particles containing the labeled target is placed in a well 46 of a flat bottom plate. The second chemiluminescent reagent is then mixed with the particles. The particles are allowed to settle to the bottom of the plate, a process that typically takes about 2 to 3 minutes.

FIG. 3 shows a system for detecting chemiluminescence. Chemiluminescence is collected through the bottom of the well 46 by a detector 48, such as a CCD camera. Optionally, an optical filter 50 is positioned between the bottom of the well 46 and the detector 48 to eliminate non-chemiluminescence light. Integration times for collecting the chemiluminescence typically range from about 1 to about 20 minutes.

FIG. 4 shows a system for exciting and collecting fluorescence. As shown in FIG. 4, a lamp 52 provides excitation light which is then optically filtered through an excitation light filter 54 to eliminate wavelengths of light not used for excitation. The filtered light is reflected by the dichroic mirror 56 and strikes the particle solution in the well 46. Emitted fluorescence from the particles transmits through the dichroic mirror 56 and fluorescence filter 58, used to eliminate any non-fluorescent light, and is collected on the detector 48. Integration times for collecting the fluorescence typically range from about 0.1 to about 20 seconds.

One skilled in the art will recognize that the fluorescent identity of the particle may be determined either before or after the detection of chemiluminescence. Once fluorescence and chemiluminescence are detected, any chemiluminescence is associated with the identity of the particle to determine the presence of one or more specific target analytes.

The catalyst is not consumed in the reaction. Therefore, in an embodiment where a catalyst is directly or indirectly attached to the target analyte and the particles are immobilized, the chemiluminescent substrate can be continuously provided to the immobilized particles, and the emitted light integrated over long periods of time (many seconds), improving signal-to-noise ratios. An increase of several orders of magnitude in integration time over convention flow cytometry can easily be achieved.

Additionally, because no illumination source is required during analyte detection, background light levels are extremely low. Moreover, because no illumination source is required to quantify the amount of analyte present, very low levels of light may be accurately measured.

Quantitation of the analyte is typically made by comparing the observed signal with those originating from calibration samples, where the concentrations are known.

### EXAMPLES

A better understanding of the present invention will be had with reference to the following examples and the figures to which they refer.

### Example 1

Two sets of 20 micron carboxylated dye-encoded beads were used. Both sets of beads were cast from a solution containing 2.4 grams of poly(styrene-co-maleic anhydride) (Aldrich Part No. 426946) dissolved in 3 mL xylene and 400 mL dichloromethane. To create a set of intensely-stained beads, 0.0024 grams of CY5 dye was added to the above solution; to create a set of dimly-stained beads, 0.0006 grams of CY5 dye was added.

Horseradish peroxidase (HRP) was directly bound to the intensely-stained bead population. This was done by first exposing the beads to a solution of EDAC/sulfo-NHS for 20 minutes, to activate the carboxyl-groups, then to HRP overnight. In this work, HRP was obtained from Sigma (Cat. No. P-6782).

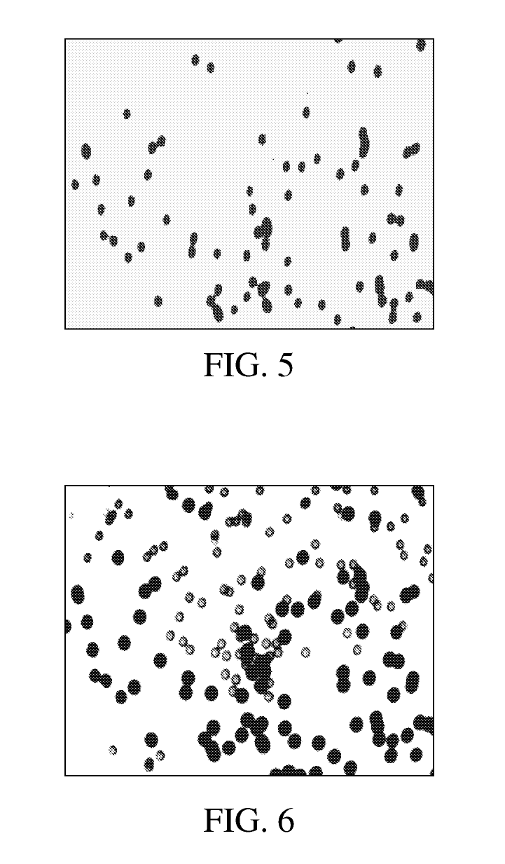

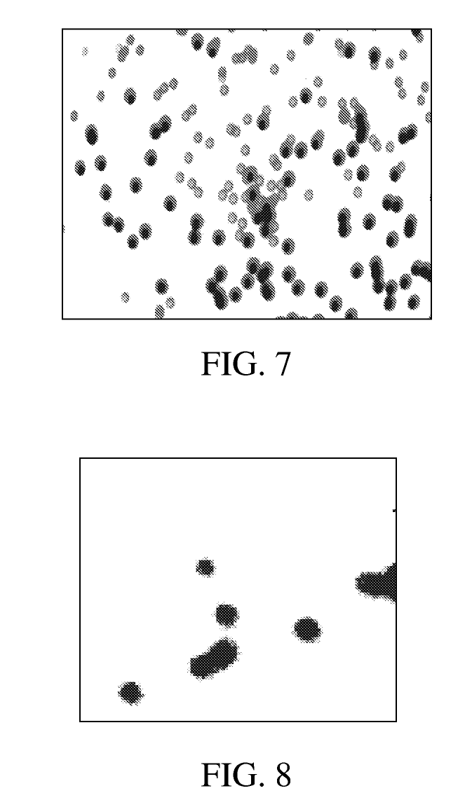

The HRP-modified intensely-stained beads were mixed with the unmodified dimly-stained beads. This sample was then mixed with 200 mL Lumigen PA-atto solution and allowed to settle to the bottom of a microtiter plate. Using the detection system shown in FIG. 3, a 120 second exposure time chemiluminescent image of the beads was taken, as shown in FIG. 5. Subsequently, the detection system shown in FIG. 4 was used to acquire a 2 second exposure time fluorescent image of the beads, as shown in FIG. 6. FIG. 7 is an overlay of the images of FIGS. 5 and 6.

As seen from FIGS. 5, 6 and 7, chemiluminescence arose only from the intensely-stained, HRP-modified bead population. This example shows that while all beads exhibit fluorescence, only beads modified to carry a first chemiluminescence component exhibit chemiluminescence.

### Example 2

Two sets of 20 micron carboxylated dye-encoded beads were used. In both cases, the beads were cast from a solution containing 2.4 grams of poly(styrene-co-maleic anhydride) (Aldrich Part No. 426946) dissolved in 3 mL xylene and 400 mL dichloromethane. To create a set of intensely-stained beads, 0.0024 grams of CY5 dye was added to the above solution; to create a set of dimly-stained beads, 0.0006 grams of CY5 dye was added.

Mouse monoclonal anti-human cardiac troponin I clone 284, a primary antibody, was covalently bound to the surface of the intensely-stained bead population by first exposing the beads to a solution of EDAC/sulfo-NHS for 20 minutes and then to the primary antibody for one hour. To create a labeled secondary antibody, HRP was bound to Mouse monoclonal anti-human cardiac troponin I clone M06 using the Pierce EZ-Link Plus activated peroxidase kit (Cat. No. 31489).

To perform the assay, 1500 intensely-stained beads with the primary antibody and 1500 dimly stained beads without the primary antibody were added to 50 microliters of troponin antigen calibrator at 100 ng/mL (Beckman Coulter Part No. 33345). This solution was incubated at room temperature with constant shaking for two hours. The sample was then centrifuged and the liquid discarded.

Fifty microliters of the HRP-labeled secondary antibody was then added to the beads, and this solution was incubated for two hours at room temperature with constant shaking. After centrifugation and decanting of the liquid, the beads were washed twice with 100 microliters of 1× PBS.

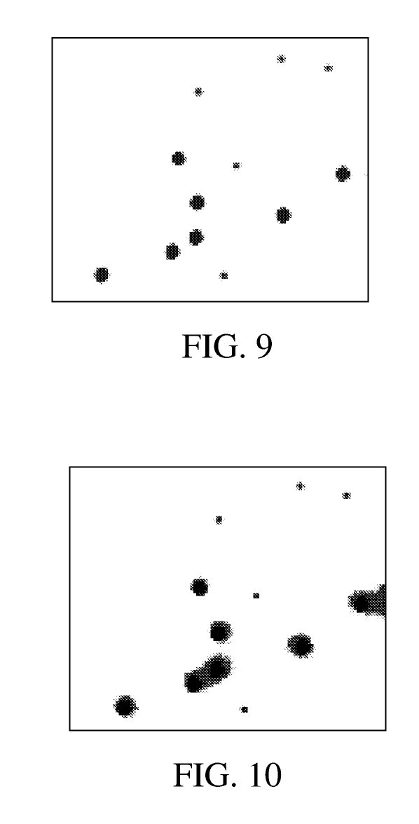

The beads were then mixed with 200 mL Lumigen PA-atto solution and allowed to settle to the bottom of a microtiter plate. Using the detection system shown in FIG. 3, a 120 second exposure time chemiluminescent image of the beads was taken, as shown in FIG. 8. Subsequently, the detection system shown in FIG. 4 was used to acquire a 2 second exposure time fluorescent image, as shown in FIG. 9. FIG. 10 is an overlay of the images in FIGS. 8 and 9.

From FIGS. 8, 9 and 10, it is clear that while all beads exhibited fluorescence, chemiluminescence arose only from the beads which captured troponin, i.e., the intensely-labeled population.

Kits

Kits comprising reagents useful for performing the methods of the invention are also provided. In one embodiment, a kit comprises at least one type of fluorescently labeled particles. Each particle type has capture probes specific to a different target bound thereto. An example of a capture probe is a primary antibody. Preferably, the kits contain multiple sets of different fluorescently labeled particles, each set having a different capture probe bindable to a particular target, to allow for multiplexing.

Additionally, the kit may also comprise a labeling reagent, bindable to any formed capture probe/target complex. An example is a secondary antibody. The labeling reagent may have a first chemiluminescence component, such as a catalyst, directly or indirectly bound thereto. Optionally, either the target analyte being sought or the labeling reagent is combinable with a reagent to generate a first chemiluminescence component and the kit comprises the reagent to generate the first chemiluminescence component on either the target analyte or the labeling reagent. In kits with multiple sets of bead populations, each bead set having different capture probes attached to them, the kits also have multiple different labeling reagent types, each labeling reagent type capable of binding with a different target in the capture probe/target complex.

Also included is a second chemiluminescence component, such as a chemiluminescent substrate, which reacts with the primary chemiluminescence component, directly or indirectly bound to the target analyte via the labeling reagent, to generate chemiluminescence. Optionally, the kit further comprises a chamber for immobilizing the particles.

Additionally, the kit may include a detecting means for detecting the fluorescence of the particles and any chemiluminescence from the presence of target analytes. The detecting means may include an excitation light source, optical manipulating devices, such as filters, lenses and mirrors, and a detector.

Having thus described the invention, it should be apparent that numerous modifications and adaptations may be resorted to without departing from the scope and fair meaning of the instant invention as set forth hereinabove and as described herein below by the claims.

Although the present invention has been described in considerable detail with reference to certain preferred versions thereof, other versions are possible. Therefore, the spirit and scope of the appended claims should not be limited to the description of the preferred versions described herein.

All features disclosed in the specification, including the claims, abstracts, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent or similar purpose, unless expressly stated otherwise. Thus, unless expressly stated otherwise, each feature disclosed is one example only of a generic series of equivalent or similar features.

Any element in a claim that does not explicitly state “means” for performing a specified function or “step” for performing a specified function, should not be interpreted as a “means” or “step” clause as specified in 35 U.S.C. § 112.

What is claimed is:

1. A method for detecting a target analyte in a sample comprising the steps of:
a. binding the target analyte to a fluorescently coded particle capable of specifically binding to the target analyte;
b. labeling the target analyte with a first chemiluminescence component;
c. adding a second chemiluminescence component to the labeled target analyte to produce chemiluminescence;
d. stabilizing the particle;
e. exciting fluorescence from the fluorescently coded particle;
f. detecting fluorescence from the fluorescently coded particle;
g. detecting the chemiluminescence.

2. The method of claim 1 wherein step (d) is performed prior to step (c).

3. The method of claim 1 wherein step (g) is performed prior to steps (e) and (f).

4. The method of claim 1 further comprising quantifying the amount of target analyte present by quantifying the chemiluminescence produced in step (g).

5. The method of claim 1 wherein either the first chemiluminescence component or the second chemiluminescence component is a catalyst.

6. The method of claim 5 wherein the catalyst comprises at least one of the group consisting of horseradish peroxidase, alkaline phosphatase, and galactosidase.

7. The method of claim 5 wherein the catalyst comprises at least one of the group consisting of Fe⁺², Fe⁺³, Cu⁺ and Cu⁺².

8. The method of claim 1 wherein the step of binding the target analyte to the fluorescently coded particle further comprises:
a. binding a capture probe to the fluorescently coded particle; and
b. binding the target analyte to the capture probe.

9. The method of claim 8 wherein the step of labeling the target analyte with a first chemiluminescence component comprises:
a. binding a labeling reagent to the target analyte; and
b. labeling the labeling reagent with a catalyst.

10. The method of claim 9 wherein the catalyst comprises at least one of the group consisting of: horseradish peroxidase, alkaline phosphatase, galactosidase, Fe⁺², Fe⁺³, Cu⁺ and Cu⁺².

11. The method of claim 1 further comprising the step of placing the particle in a microtiter plate; and
wherein the particle is stabilized by allowing the particle to settle on the bottom of the microtiter plate.

12. A kit for detecting a target analyte in a sample, the kit comprising:
a. a plurality of fluorescently coded particles, each particle having at least one capture probe bound thereto, each capture probe being bindable to the target analyte;
b. a labeling reagent bindable to the target analyte, the labeling reagent comprising a first chemiluminescence component; and
c. a second chemiluminescence component reactable with a first chemiluminescence component directly or indirectly bound to the target analyte to generate chemiluminescence.

13. The kit of claim 12 wherein the first chemiluminescence component comprises a catalyst.

14. The kit of claim 12 wherein the catalyst is at least one of the group consisting of: horseradish peroxidase, alkaline phosphatase, galactosidase, Fe⁺², Fe⁺³, Cu⁺ and Cu⁺².

15. The kit of claim 12 further comprising a chamber for immobilizing the particles.

16. The kit of claim 12 further comprising a means for detecting fluorescence from the particles and chemiluminescence from the presence of the target analyte.

17. The kit of claim 16 wherein the means for detecting comprises:
an excitation light source; and
a CCD.

18. A kit for detecting a plurality of target analytes in a sample, the kit comprising:
a. a plurality of sets of fluorescently coded particles, each set comprising particles having at least one capture probe bound thereto, and each set having a capture probe capable of binding with a different target analyte;
b. a plurality of different labeling reagents bindable to the target analytes, the labeling reagents being labeled with a first chemiluminescence component; and
c. a second chemiluminescence component reactable with the first chemiluminescence component bound to the target analyte to generate chemiluminescence.

19. A method of detecting a plurality of target analytes comprising the steps of:
a. obtaining the kit of claim 18;
b. adding a sample containing the target analytes at conditions such that the target analytes in the sample bind to the capture probes bound to the particles to form capture probe/target complexes;
c. following step (b), adding the labeling reagents at conditions such that the labeling reagents bind to the primary probe/target complexes; and
d. adding the second chemiluminescence component to generate chemiluminescence.

20. A method for detecting a plurality of different target analytes in a sample comprising the steps of:
a. binding each of the target analytes to a respective particle specific to the target analyte, each particle being fluorescently coded for identification;
b. labeling each of the target analytes with a first chemiluminescence component;
c. adding a second chemiluminescence component to the labeled target analytes to produce chemiluminescence;
d. stabilizing the particles;
e. exciting fluorescence from the fluorescently coded particles;
f. detecting fluorescence from the fluorescently coded particles;
g. detecting the chemiluminescence; and
h. associating detected fluorescence with detected chemiluminescence to determine the presence of one or more of the target analytes.

21. The method of claim 20 further comprising the step of immobilizing the fluorescently coded particles prior to step (e).

22. The method of claim 20 wherein step (g) is performed prior to steps (e) and (f).

23. The method of claim 20 further comprising quantifying the amount of at least one of the plurality of target analytes present by quantifying the chemiluminescence produced in step (g).

24. The method of claim 20 wherein the first chemiluminescence component is a catalyst comprising at least one of the group consisting of horseradish peroxidase, alkaline phosphatase, galactosidase, Fe⁺², Fe⁺³, Cu⁺ and Cu⁺².

25. The method of claim 20 wherein the step of binding each of the target analytes to a particle specific to the target analyte further comprises:
a. binding a target analyte specific capture probe to the fluorescently coded particle; and
b. binding the target analyte to the capture probe.

26. The method of claim 20 wherein the step of labeling each of the target analytes with a first chemiluminescence component comprises:
a. binding a target analyte specific labeling reagent to the target analyte; and
b. labeling the labeling reagent with at least one of the group consisting of: horseradish peroxidase, alkaline phosphatase, galactosidase, Fe⁺², Fe⁺³, Cu⁺ and Cu⁺².

27. A method for detecting a target analyte in a sample comprising the steps of:
a. mixing the target analyte with a plurality of fluorescently coded particles bindable to the target analyte;
b. labeling the fluorescently coded particles having no target analyte with a first chemiluminescence component;
c. adding a second chemiluminescence component to the labeled fluorescently coded particles to produce chemiluminescence;
d. stabilizing the particles;
e. exciting fluorescence from the fluorescently coded particle;
f. detecting fluorescence from the fluorescently coded particle;
g. detecting the chemiluminescence.
